(19) United States
(12) Patent Application Publication
(10) Pub. No.: US 2011/0172677 A1
(43) Pub. Date: Jul. 14, 2011

(54) Medical instrument positioning device

(76) Inventor: Daniel Clemens Newman, Wilmette, IL (US)

(21) Appl. No.: 12/655,944

(22) Filed: Jan. 11, 2010

(51) Int. Cl. A61F 11/00

(52) U.S. Cl. 606/108

(57) ABSTRACT

A medical instrument positioning device that allows medical personnel to quickly and easily insert and remove medical instruments and accessories through a portal in a patient is described. The device guides the inserted instruments directly to the surgical site so that the surgeon does not have to search to reacquire the site. The device is generally “L” shaped, having a handle on the proximal end and a somewhat sharpened tip on the distal end. The body of the device is generally curved or otherwise shaped to form a channel down which medical instruments, cameras, accessories, etc. can be positioned. The tip of the device is shaped so as to be easily inserted into a portal. Once inserted, the device is positioned so that the distal end is in proximity to a surgical site.

### TECHNICAL FIELD

This invention relates generally to the field of medical instruments and in particular to a medical instrument positioning device.

### BACKGROUND

In recent years, minimally invasive medical procedures have become extremely popular because of the many benefits to the patient, including, improved recovery time and reduced pain as compared to conventional open medical procedures. Minimally invasive medical procedures use one or more access openings or portals in a patient's body. A physician, surgeon, or other medical personnel can use a portal to position medical devices such as instruments, cameras, and accessories to perform any number of medical procedures. One such common instrument is an arthroscope, including, without limitation, laparoscopes. Various types of medical devices are described in detail in U.S. Pat. Nos. 5,040,715, 5,084,057, 5,100,420, 5,171,247, and 5,176,695. Arthroscopy is a term used to describe an “endosurgical” approach using an arthroscope (or a rigid laparoscope). In these types of procedures, a rigid access tube or cannula is often placed into a portal in order to keep the portal open and allow medical devices to be inserted and removed without damage to tissues surrounding the portal site.

Although cannulas provide many benefits, they have some limitations. In particular, when an instrument is removed from or inserted into a cannula, the cannula can shift or move causing it to be difficult to reacquire the exact surgical site after a second instrument is inserted in the cannula. Even if the cannula itself does not shift when an instrument is removed or inserted, the patient's tissues, fluids, etc. often reposition themselves causing the surgeon to have to reacquire the surgical site after inserting the new instrument. Furthermore, since cannulas have a fixed diameter, some devices are unable to fit within some cannulas.

Another common problem associated with arthroscopies is the relatively high cost of medical devices used in such procedures. Given their high costs, many medical offices, hospitals, etc. limit the number of duplicative instruments on hand and so a surgeon is often required to move a single instrument between various access portals repeatedly, compounding the problems discussed above.

A number of variations and advancements have been made to cannulas, see U.S. Pat. Nos. 5,009,643, 5,122,122, and 5,364,367. However, although these variations deal with some of the deficiencies associated with cannulas, they are not entirely successful in addressing the above problems. Therefore, a need exits for an inexpensive device that can assist a physician in inserting and removing various medical instruments through portals while providing quick access to the same surgical sites with each succeeding instrument.

### SUMMARY

Embodiments of the present invention described and claimed herein address the foregoing limitations and problems by providing a medical instrument positioning device. The device allows a surgeon to quickly and easily insert and remove medical instruments and accessories through a portal in a patient. The device guides the inserted instruments directly to the surgical site so that the surgeon does not have to search to reacquire the site. The device is generally “L” shaped, having a handle on the proximal end and a somewhat sharpened tip on the distal end. The body of the device is generally curved or otherwise shaped to form a channel down which medical instruments, cameras, accessories, etc. can be positioned. The tip of the device is shaped so as to be easily inserted into a portal. Once inserted, the device is positioned so that the distal end is in proximity to a surgical site. In one embodiment, the device is utilized with a cannula. In an alternate embodiment, the device is utilized without a cannula.

One method for using the device is as follows. A cannula is placed within a portal and a medical instrument is inserted into the cannula. A surgical site is selected and a procedure is performed. Then, a medical instrument positioning device is inserted between the medical instrument and the portal wall and positioned such that the distal end of the device is in proximity to the surgical site. The medical instrument is then removed and either a new instrument is selected or the original instrument is chosen for insertion. In either case, the instrument is placed against the device near the proximal end and slid down the device towards the distal end. The medical instrument is thereby placed in the same position as the original instrument and the medical instrument positioning device can then be removed or left in place to be used if another change of instruments is required.

The present invention provides many benefits over the prior art. Because the device provides a positioning channel rather than a tube, it can accommodate various sizes of medical instruments. Furthermore, the device can be used with medical instruments having an integrated cannula, or the cannula can be separate from the instrument. As the device also addresses some of the problems for which a cannula normally is required, the device can even be used without a cannula. Additionally, and perhaps of primary importance, the device allows a physician to quickly and easily swap-out medical instruments without having to search to reacquire the specific surgical location within the patient.

### BRIEF DESCRIPTION OF THE DRAWINGS

The aforementioned and other features and objects of the present invention and the manner of attaining them will become more apparent and the invention itself will be best understood by reference to the following descriptions of a preferred embodiment and other embodiments taken in conjunction with the accompanying drawings, wherein:

FIG. 1 illustrates a perspective view of an exemplary embodiment of a medical instrument positioning device near a portal and in proximity to a medical instrument.

FIG. 2 illustrates a side view of an exemplary embodiment of a medical instrument positioning device.

FIG. 3 illustrates a front cross-sectional view of an exemplary embodiment of a medical instrument positioning device.

FIG. 4 illustrates exemplary operations for a method of using a medical instrument positioning device.

### DETAILED DESCRIPTION

In one embodiment, a medical instrument positioning device is generally “L” shaped, having a handle on the proximal end and a somewhat sharpened insertion tip on the distal end. The handle can have various shapes, and extends roughly perpendicular to the main body of the device. The body of the device is generally curved to form a channel down which medical instruments, cameras, accessories, etc. can be positioned. In order to ease the insertion process, the distal end of the device can be somewhat sharpened so that it more easily slides into a portal either between a medical instrument and a cannula or between a medical instrument and the tissues of the portal itself. Once inserted, the device is positioned so that the distal end is in proximity to a surgical site.

an insertion tip extending from the second end of the channel and adapted to be inserted into a portal in a patient.

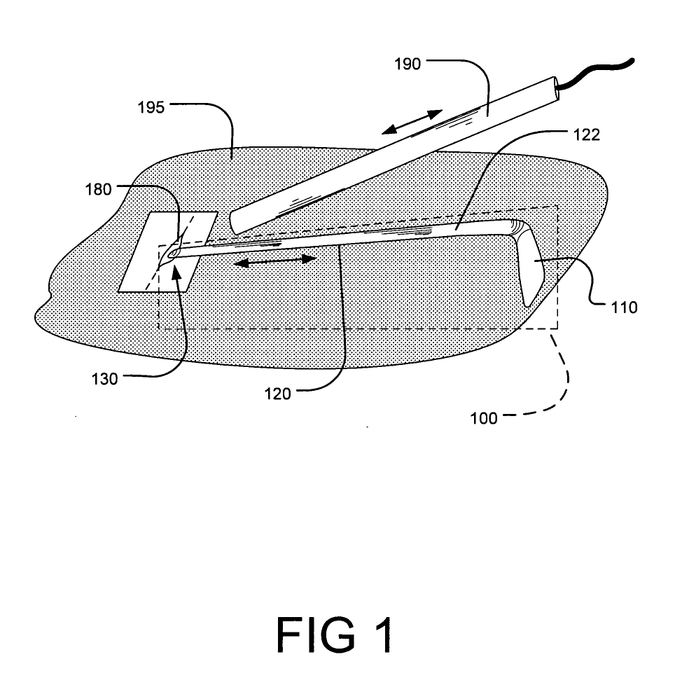

FIG. 1 illustrates a perspective view of an exemplary embodiment of a medical instrument positioning device 100 near a portal 180 and in proximity to a medical instrument 190. The device comprises a handle 110, a channel 120, and an insertion tip 130.

The handle 110 is shown in the embodiment of FIG. 1 as being generally flat and adapted to provide a surgeon or other medical personnel a good grip. In other embodiments, other handle 110 shapes are contemplated. For example, the handle 110 can be ergonomically shaped to more closely fit the hand or fingers manipulating it. Also shown in FIG. 1 is a standard surgical drape 195.

As illustrated in FIG. 1, the channel 120 is roughly cylindrical in shape, having a proximal end attached to the handle 110 and a distal end attached to the insertion tip 130. The channel 120 is adapted to receive a medical instrument 190 or other accessory against the channel 120 and allow it to be slid up or down the channel 120. The center of the channel 120 can be described as a spine extending between the two ends, and thus forming a trough-shape. A cross-section taken perpendicular to the spine would expose a section of an arc that extends only approximately 90 to 180 degrees versus the full 360 degree circular cross-section of a cylinder. In other embodiments, the channel 120 can have a cross-section that extends through less than 90 degrees or more than 180 degrees of an arc. Furthermore, the channel 120 can have other cross-sectional shapes. For example, the channel 120 can have a cross-section shaped like a “v” or a “u” in another embodiment.

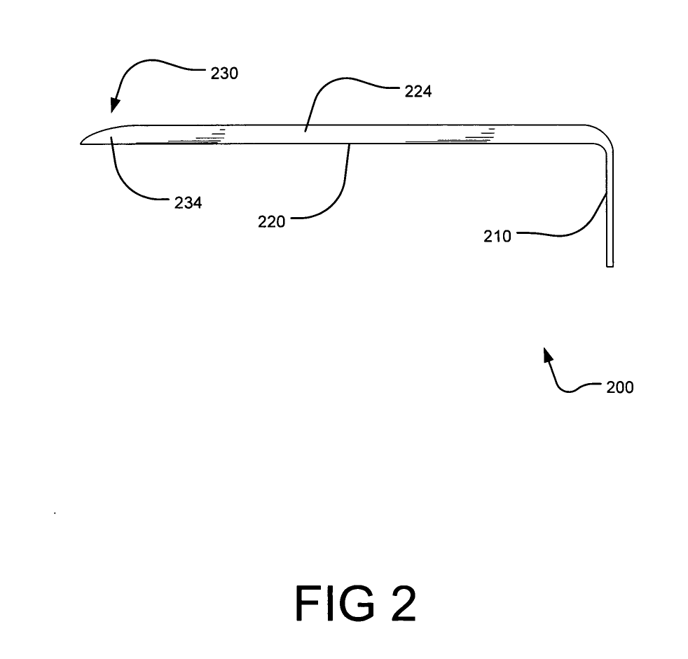

As shown in FIG. 1, the device 100 is placed such that a medical instrument 190 can be positioned against the device 100. Once positioned, the channel interior wall 122 can guide the medical instrument 190 as it is removed or inserted into the portal 180. Thus, the channel interior wall 122 is shaped to readily accept the various medical instruments 190 used in laparoscopy and related surgeries. The channel exterior wall (not illustrated in FIG. 1, see FIG. 2, exterior wall 224) slides along the sidewalls of a cannula or against the portal 180 itself as the device 100 is inserted or removed from the portal 180.

The insertion tip 130 illustrated in FIG. 1 is somewhat sharpened to enhance the ease of insertion of the medical instrument positioning device 100 into the portal 180. The tip 130 can be sharpened by having material removed from the tip interior wall (not illustrated in FIG. 1), from the tip exterior wall (not illustrated in FIG. 1, see FIG. 2, tip exterior wall 234), or from both walls. It is preferable that the tip 130 not be overly sharpened as this could result in a cutting edge that might cause unwanted damage to the tissues of the portal 180 or those in proximity to the surgical site within the patient.

FIG. 2 illustrates a side view of an exemplary embodiment of a medical instrument positioning device 200. The device comprises a handle 210, a channel 220, and an insertion tip 230. The handle 210 is located at the proximal end of the device 200. As shown in the embodiment of FIG. 2, the handle 210 extends generally perpendicular from the channel 220. In other embodiments, the handle 210 can extend at other angles relative to the channel 220.

The insertion tip 230 is located at the distal end of the device 200. The shape of the insertion tip 230 in the embodiment shown in FIG. 2 varies slightly from the embodiment shown in FIG. 1. Such variations can be made without departing from the scope of the invention.

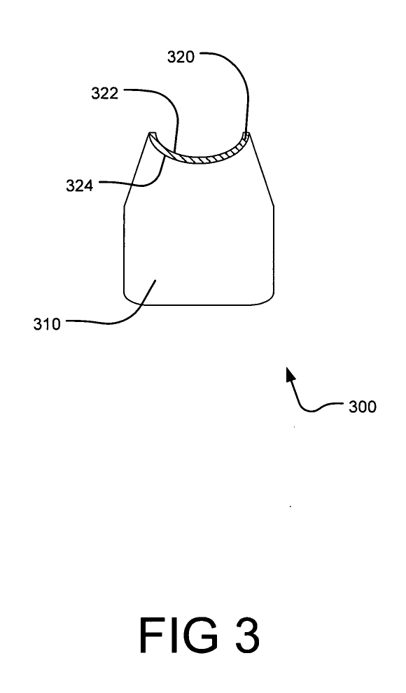

FIG. 3 illustrates a front, cross-sectional view of an exemplary embodiment of a medical instrument positioning device 300. In this view, the cross-sectional shape of the channel 320 is apparent. As discussed above, other shapes for the channel 320 are contemplated. As shown in FIG. 3, the channel interior wall 322 is shaped to readily accept medical instruments, cameras, and accessories commonly used in arthroscopies/endoscopies. In other embodiments, other shapes for the interior wall 322 are contemplated in order to more readily accept the various shaped medical devices that can be used. As shown in FIG. 3, the channel exterior wall 324 is shaped to easily slip between a cannula or portal opening and a medical device. If a non-round cannula or non-standard portal is used, the exterior wall 324 can be shaped accordingly in alternate embodiments.

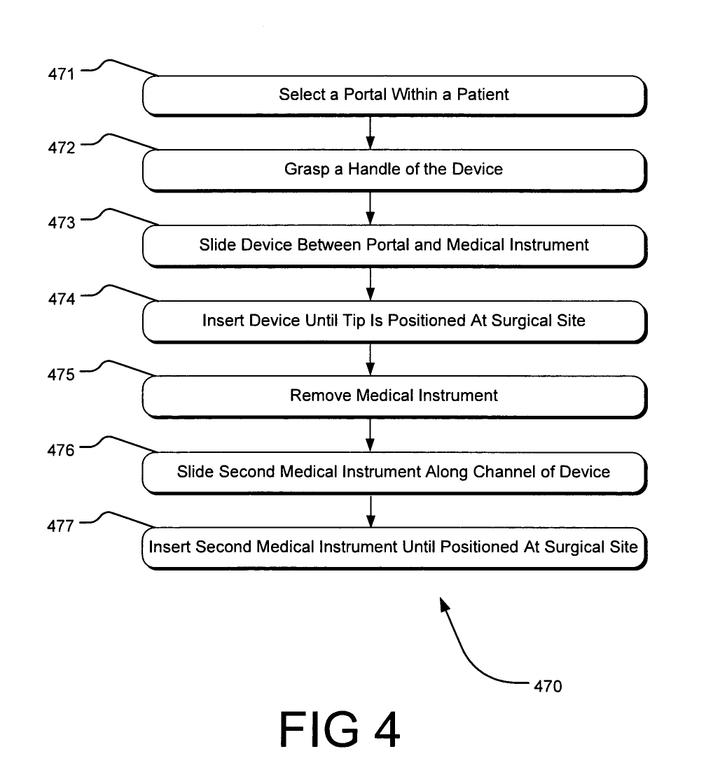

FIG. 4 illustrates exemplary operations 470 for a method of using a medical instrument positioning device as described above. The operations involve selecting a portal within a patient 471, grasping the handle of the device 472, sliding the device between the portal and the medical instrument 473, inserting the device until the tip is positioned at the surgical site 474, removing the medical instrument 475, sliding a second medical instrument along the channel of the device 476, and inserting the second medical instrument until it is positioned at the surgical site 477.

The “Select a Portal Within a Patient” Operation 471 involves the surgeon or other medical personnel choosing a portal in a patient upon which to perform the method. The portal should already have a first medical instrument (or other medical accessory) in use at a surgical site within the patient.

The “Grasp a Handle of the Device” Operation 472 involves the surgeon grasping the handle of the medical instrument positioning device. As described above, the handle is a generally flat component extending from the proximal end of the device and adapted for being gripped by a surgeon in order to maneuver the device into position.

The “Slide Device Between Portal and Medical Instrument” Operation 473 involves the surgeon positioning the channel of the device against the first medical instrument and then sliding the device between a wall of the portal and the first medical instrument.

The “Insert Device Until Tip Is Positioned At Surgical Site” Operation 474 involves the surgeon sliding the device along the medical instrument and into the patient. The surgeon should stop inserting the device when the insertion tip is positioned near the distal end of the first medical instrument and at the surgical site.

The “Remove Medical Instrument” Operation 475 involves the surgeon removing the first medical instrument from the patient by sliding the medical instrument along the channel of the device until it has completely cleared the portal. The first medical instrument is then available to be used in a second portal within the patient. Alternatively, the first instrument can simply be set aside.

The “Slide Second Medical Instrument Along Channel of Device” Operation 476 involves the surgeon placing a second medical instrument or other medical accessory against the channel of the device. The surgeon then slides the instrument down the channel and the channel guides the distal end of the second medical instrument into the portal.

The “Insert Second Medical Instrument Until Positioned At Surgical Site” Operation 477 involves the surgeon continuing to slide the instrument along the channel of the device. The medical instrument positioning device guides the second medical instrument directly to the surgical site, at which time the surgeon stops inserting the second medical instrument and can begin using the second medical instrument. The surgeon may choose to remove the medical instrument positioning device before beginning to use the second medical instrument.

The above specification, examples and data provide a description of the structure and use of exemplary embodiments of the described articles of manufacture and methods. Many embodiments can be made without departing from the spirit and scope of the invention.

What is claimed is:

1. A medical instrument positioning device, comprising:
a channel adapted for slidably receiving a medical instrument or medical accessory, wherein the channel has a first end, a second end and a spine extending between the first end and the second end;
a generally flat handle adapted for gripping, and extending from the first end of the channel at an angle from the spine; and
an insertion tip adapted for slidably receiving a medical instrument or medical accessory, extending from the second end of the channel, and adapted to be inserted into a portal in a patient.

2. The medical instrument positioning device of claim 1, wherein a cross-section of the channel taken perpendicular to the spine is generally cylindrical in shape, having an arc that spans between 45 degrees and 180 degrees.

3. The medical instrument positioning device of claim 1, wherein a cross-section of the channel taken perpendicular to the spine is generally cylindrical in shape, having an arc that spans more than 180 degrees.

4. The medical instrument positioning device of claim 1, wherein a cross-section of the channel taken perpendicular to the spine is generally cylindrical in shape, having an arc that spans less than 45 degrees.

5. The medical instrument positioning device of claim 1, wherein a cross-section of the channel taken perpendicular to the spine is generally V-shaped.

6. The medical instrument positioning device of claim 1, wherein a cross-section of the channel taken perpendicular to the spine is generally U-shaped.

7. A medical instrument positioning device, comprising:
a channel adapted for slidably receiving a medical instrument or medical accessory, wherein the channel has a first end, a second end and a spine extending between the first end and the second end;
a generally flat handle adapted for gripping, and extending from the first end of the channel at an angle of between 60 degrees and 120 degrees from the spine; and
a somewhat sharpened insertion tip adapted for slidably receiving a medical instrument or medical accessory, extending from the second end of the channel, and adapted to be inserted into a portal in a patient.

8. The medical instrument positioning device of claim 7, wherein a cross-section of the channel taken perpendicular to the spine is generally cylindrical in shape, having an arc that spans between 45 degrees and 180 degrees.

9. The medical instrument positioning device of claim 7, wherein a cross-section of the channel taken perpendicular to the spine is generally cylindrical in shape, having an arc that spans more than 180 degrees.

10. The medical instrument positioning device of claim 7, wherein a cross-section of the channel taken perpendicular to the spine is generally cylindrical in shape, having an arc that spans less than 45 degrees.

11. The medical instrument positioning device of claim 7, wherein a cross-section of the channel taken perpendicular to the spine is generally V-shaped.

12. The medical instrument positioning device of claim 7, wherein a cross-section of the channel taken perpendicular to the spine is generally U-shaped.

13. A method of using a medical instrument positioning device as claimed above, the method comprising:
selecting a portal in a patient, the portal having a first medical instrument already in use at a surgical site within the patient;
grasping a handle of the device;
sliding the device between a wall of the portal and the first medical instrument;
inserting the device into the patient until an insertion tip of the device is in proximity to a distal end of the first medical instrument so as to position the insertion tip at the surgical site;
removing the first medical instrument from the patient;
sliding a second medical instrument along a channel in the device, the channel guiding a distal end of the second medical instrument into the patient;
inserting the second medical instrument into the patient until the distal end of the second medical instrument is positioned at the surgical site.
